(19) United States
(12) Patent Application Publication
(10) Pub. No.: US 2024/0374374 A1
(43) Pub. Date: Nov. 14, 2024

(54) Intraocular Add-On Lens for Correction of Refraction and Extended Depth of Field

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 18/690,375

(22) PCT Filed: Sep. 9, 2022
(86) PCT No.: PCT/NL2022/050512

§ 371 Date: Mar. 8, 2024

(30) Foreign Application Priority Data
Sep. 10, 2021 (NL) 2029167

(51) Int. Cl. A61F 2/16

(52) U.S. Cl. CPC A61F 2/1618; A61F 2/1627; A61F 2/1648; A61F 2002/1681; A61F 2250/0008

(57) ABSTRACT

An intraocular lens for correcting of refraction, includes at least one optical lens with an optical axis; and haptics, connected to the optical lens, for positioning the lens in front of the capsular bag of an eye, for example arranged to be placed in the sulcus of the eye; wherein haptics include at least one ring shaped flexible arm, preferably at least a pair of ring shaped flexible arms arranged on opposite sides of the optical lens, preferably as seen perpendicular to the optical axis. The optical lens includes an optical power element for correcting the refractive power of an eye. The optical lens further includes a multifocal optical surface for extending the depth of field of an eye.

### CROSS REFERENCE TO RELATED APPLICATIONS

This application is the United States national phase of International Patent Application No. PCT/NL2022/050512 filed Sep. 9, 2022, and claims priority to The Netherlands Patent Application No. 2029167 filed Sep. 10, 2021, the disclosures of which are hereby incorporated by reference in their entireties.

### BACKGROUND OF THE INVENTION

### Field of the Invention

Intraocular add-on lens to be positioned in front of the capsular bag, at the sulcus plane of the eye.

### Description of Related Art

The invention is related to flexible haptics to provide positioning the lens in between the natural lens of the eye and the iris of the eye largely independent of the diameter of the sulcus, and, an optical lens to provide correction of the refractive power of the eye and a multifocal optical surface to provide extended depth of field which can be an adjustable extended depth of field.

### SUMMARY OF THE INVENTION

This document discloses an intraocular add-on lens for correction of refraction and extended depth of field. The intraocular lens comprises at least one optical lens for optical functionality and haptics, anchors, for mechanical functionality, meaning: anchoring the intraocular lens in the eye.

The lens is to be positioned in front of the capsular bag, at the sulcus plane of the eye. The lens comprises:
at least one ring shaped flexible haptics (as in, for example disclosed in NL2015538, incorporated herein by reference) to provide positioning the lens in between the natural lens of the eye and the iris of the eye largely independent of the diameter of the sulcus, and,
an optical lens comprising at least one optical lens element with the optical lens providing correction of the refractive power of the eye which correction is, in general, a correction of negative optical power for myopic eyes, and,
the optical lens can also have aspheric optical power and/toric optical power corrections, and,
the optical lens comprises also at least one multifocal optical surface to provide extended depth of field to correct for, for example, presbyopia of the eye.

The invention in particular provides an intraocular lens for correcting of refraction, comprising: at least one optical lens with an optical axis; and haptics, connected to the optical lens, for positioning the lens in front of the capsular bag of an eye, for example arranged to be placed in the sulcus of the eye; wherein haptics comprise at least one ring shaped flexible arm, preferably at least a pair of ring shaped flexible arms arranged on opposite sides of the optical lens, preferably as seen perpendicular to the optical axis; wherein the optical lens comprises an optical power element for correcting the refractive power of an eye; and wherein the optical lens further comprises a multifocal optical surface for extending the depth of field of an eye. The haptics are typically provided at the sides edges of the optical lens, and are used to position the lens in the eye such that the optical lens can be aligned with the natural lens of the eye and the haptics are used to keep the intraocular lens in place.

The optical power element and the multifocal optical surface are for example arranged on opposite sides of the optical lens, preferably as seen along the optical axis. These opposite sides are seen as opposite when looking along the optical axis of the eye. One of these is thus in front and one at the back.

The multifocal optical surface may be a cubic optical surface, or a chiral optical surface. A cubic optical surface may be a surface which can be defined by cubic polynomials. Such surfaces are typically curved and not flat and the optical power of such lens thus depends on the positioning along the optical surface. For chiral surfaces it is typically the rotation of the surfaces which provides a changing optical power of a lens.

The optical lens may comprise at least two optical elements, wherein each optical element is fitted with a multifocal optical surface to adjust the optical properties of the intraocular lens by adjustment of the mutual position of the optical elements.

The multifocal optical surface can be cubic optical surface, or, alternatively, a chiral optical surface as disclosed in WO2011102719, also incorporated herein by reference, or, alternatively, any stepwise or gradual multifocal optical surface, or, alternatively, a combination of any multifocal/extended depth of field surfaces.

The lens can have at least two optical elements with each element fitted with a multifocal optical surface, for example, a chiral optical surface. Translation of the mutual position, for example, mutual rotation, of the surfaces can change the multifocal power, or, alternatively, the distance between the multifocal spots, or, alternatively, the power of extended depth of field which will provide an intraocular add-on lens of which optical properties are, for example, surgically, adjustable once in the eye, or, alternatively, are adjustable by the ciliary muscle of the eye by the wearer of the intraocular lens.

The optical lens may define an optical plane, wherein the haptics are positioned tilted compared to the optical plane or the optical lens, in particular tilted towards the multifocal optical surface of the optical lens. The direction in which at least one of the haptics extends and the direction the optical plane or the optical lens extend may enclose an angle, in particular an angle between 100 and 160 degrees, in particular between 110 and 150 degrees, more in particular between 120 and 140 degrees.

In other words, the invention may further relate to an intraocular add-on lens for correction of refraction and to provide extended depth of field with the lens comprising at least one optical lens and haptics which lens is adapted to be positioned in front of the capsular bag, at the sulcus plane of the eye, wherein the lens comprises at least one ring shaped flexible haptics to provide positioning of the lens in between the natural lens of the eye and the iris of the eye; an optical lens comprising at least one optical lens element to provide correction of the refractive power of the eye with the optical lens comprising also at least one multifocal optical surface to provide extended depth of field.

### BRIEF DESCRIPTION OF THE DRAWINGS

The terms Fig., Figs., Figure, and Figures are used interchangeably in the specification to refer to the corresponding figures in the drawings.

Further advantages, features, and details of the invention are elucidated on the basis of preferred embodiments thereof, wherein reference is made to the accompanying drawings, in which:

FIG. 1 schematically shows a top view of a lens according to the invention;

FIG. 2 schematically shows a side view of a lens according to the invention; and

FIG. 3 schematically shows a side view of a lens according to the invention, when placed in the eye as add-on lens.

### DESCRIPTION OF THE INVENTION

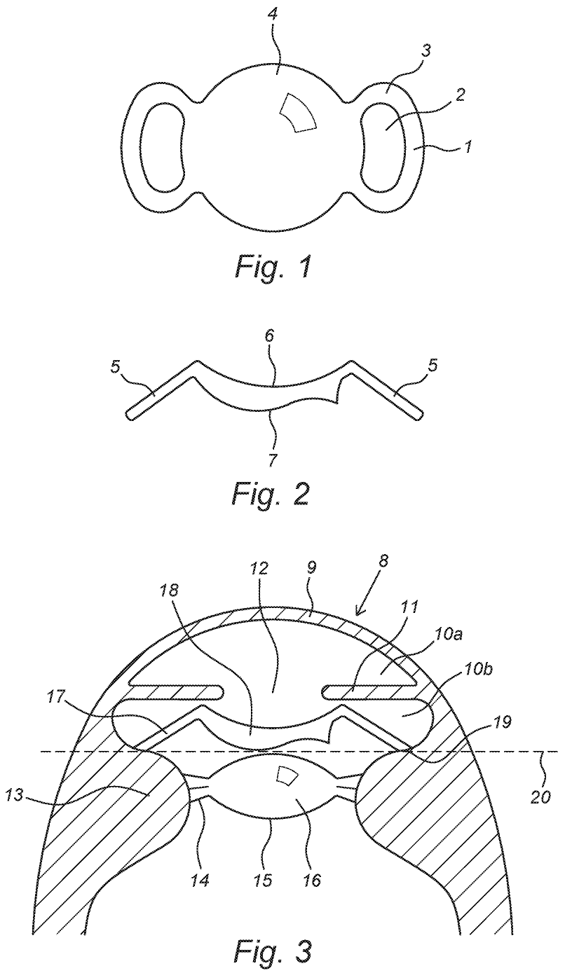

FIG. 1 shows, in top view, the intraocular add-on lens, with, in this example, the ring shaped haptic with a rim, 1, and a largely ring-shaped opening, 2, the section of the elastic hinge, 3, and the optical lens, 4.

FIG. 2 shows, in side view, the intraocular lens, with, in this example the backward tilted haptics, 5, to have the intraocular lens following the curvature of the space in between the iris and the natural lens, and, in this example, a negatively powered lens surface, 6, to correct a myopic eye and an optical multifocal/extended depth of field optical surface, 7, in this example a cubic optical surface.

FIG. 3 shows the intraocular add-on lens in the eye, 8, with the eye having a cornea, 9, an anterior chamber, 10a, and posterior chamber, 10b, an iris, 11, and a pupil, 12, a ciliary mass, 13, zonulae, 14 and a capsular bag, 15, containing the natural lens, 16. The intraocular add-on lens with the haptics, 17 and the optical lens, 18, is positioned in between the natural lens and the iris, at the sulcus, 19, at the sulcus plane, 20.

1. An intraocular lens for correcting of refraction, comprising:
a) at least one optical lens with an optical axis; and
b) haptics, connected to the optical lens, for positioning the lens in front of the capsular bag of an eye, for example arranged to be placed in the sulcus of the eye;
c) wherein haptics comprise at least a pair of ring shaped flexible arms arranged on opposite sides of the optical lens as seen perpendicular to the optical axis;
d) wherein the optical lens comprises an optical power element for correcting the refractive power of an eye; and
e) wherein the optical lens further comprises a multifocal optical surface for extending the depth of field of an eye.

2. The intraocular lens according to claim 1, wherein the optical power element and the multifocal optical surface are arranged on opposite sides of the optical lens, preferably as seen along the optical axis.

3. The intraocular lens according to claim 1, wherein the multifocal optical surface is a cubic optical surface.

4. The intraocular lens according to claim 1, wherein the multifocal optical surface is a chiral optical surface.

5. The intraocular lens according to claim 1, wherein the optical lens comprises at least two optical elements, wherein each optical element is fitted with a multifocal optical surface to adjust the optical properties of the intraocular lens by adjustment of the mutual position of the optical elements.

6. The intraocular lens according to claim 1, wherein the optical lens defines an optical plane, wherein the haptics are positioned tilted compared to the optical plane or the optical lens, in particular tilted towards the multifocal optical surface of the optical lens.
